(19) United States
(12) Patent Application Publication
(10) Pub. No.: US 2010/0137795 A1
(43) Pub. Date: Jun. 3, 2010

(54) INTRA-PERICARDIAL PROTECTIVE DEVICE

(76) Inventor: Da-Li Feng, Rochester, MN (US)

Correspondence Address: FISH & RICHARDSON P.C., PO BOX 1022, MINNEAPOLIS, MN, 55440-1022, US

(21) Appl. No.: 12/699,637

(22) Filed: Feb. 3, 2010

Related U.S. Application Data
(62) Division of application No. 11/752,776, filed on May 23, 2007, now abandoned.
(60) Provisional application No. 60/803,444, filed on May 30, 2006.

(51) Int. Cl. A61M 25/10

(52) U.S. Cl. 604/96.01

(57) ABSTRACT

Devices and methods that can be used to protect the esophagus during procedures such as atrial ablation. The devices can include an elongate tubular member that defines one or more lumens extending therethrough. The devices also can have a balloon attached at or near an end of the elongate tubular member. The balloon can partially or completely surround the outer surface of a segment of the elongate tubular member, and can be in fluid communication with a lumen of the elongate tubular member.

### CROSS-REFERENCE TO RELATED APPLICATIONS

This application is a divisional of U.S. application Ser. No. 11/752,776, filed May 23, 2007, which claims benefit of priority from U.S. Provisional Application Ser. No. 60/803,444, filed May 30, 2006.

### TECHNICAL FIELD

This document relates to intra-pericardial devices, and more particularly to intra-pericardial devices that can be used to protect the esophagus during atrial fibrillation ablation procedures.

### BACKGROUND

Atrial fibrillation is a disorder that occurs in about 3 to 5 percent of people over the age of 65, and is the most common cause of arrhythmia in humans. During atrial fibrillation, the atria of the heart quiver instead of beating effectively. Since blood is not pumped completely out of the atria, it may pool and clot, increasing the likelihood of stroke.

Delivery of radiofrequency pulses around the pulmonary veins can be used as a nonpharmacologic approach to treatment of patients with drug-refractory atrial fibrillation. Circumferential ablation of atrial tissue surrounding the pulmonary veins can be more effective than restricted segmental ablation at the pulmonary vein ostia. During such procedures, high-energy (60-85 W) radiofrequency pulses can be applied through a distal tip catheter to create deep left atrial lesions.

### SUMMARY

A risk associated with creating deep lesions on the left atrial posterior wall is esophageal damage and consequent development of left atria-esophageal fistula. Such a complication often is fatal. The present document provides devices that can be used to protect the esophagus from damage during atrial ablation (e.g., circumferential atrial ablation), and methods for using such devices.

In one aspect, this document features a method for protecting the esophagus during an atrial ablation procedure. The method can include inserting a balloon into the pericardial space between the esophagus and the heart of a patient in need of an atrial ablation procedure, and inflating the balloon, wherein the balloon contains an echogenic or X-ray transparent material. The balloon can be attached to a catheter. The balloon can be in fluid communication with a lumen of the catheter. The balloon can be inflated with air, water, saline, or gel. The balloon can have a length between about 3 cm and about 5 cm. The balloon, when not inflated, can have a width between about 2 cm and about 4 cm. The balloon, when inflated, can have a diameter from about 0.5 cm to about 1 cm. The balloon can have a thickness between about 0.3 cm and about 1.2 cm. The balloon can be made of a material capable of withstanding heat of at least 50° C.

In another aspect, this document features a device having a catheter with a first end and a second end, a first lumen extending from the first end to the second end, a second lumen extending from the first end to a position proximate the second end, and a balloon surrounding a segment of the catheter proximate the second end. The balloon can be in fluid communication with the first end via the second lumen. The balloon can contain an echogenic or X-ray transparent material. The device can have an overall length between about 30 cm and about 50 cm. The balloon can have a length between about 2 cm and about 6 cm, a width between about 2 cm and about 4 cm, and/or a thickness between about 0.3 cm and about 1.2 cm. The balloon can be made of a material capable of withstanding heat of at least 50° C.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing the proximity of the esophagus to the left atrium of the heart.

FIG. 2 is a side view of a device that can be used to protect the esophagus.

FIG. 3 is a cross-sectional view of a device that can be used to protect the esophagus.

FIG. 4 is an end view of a device that can be used to protect the esophagus.

Like reference symbols in the various drawings indicate like elements.

### DETAILED DESCRIPTION

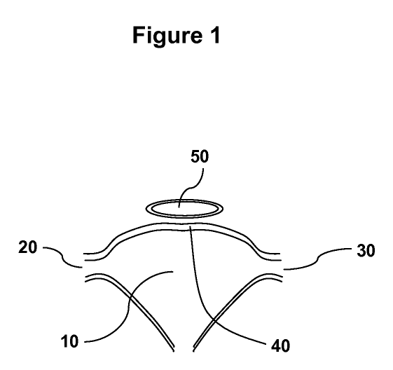

This document provides devices and methods that can be used to protect the esophagus from damage that can occur, for example, during an atrial ablation procedure. FIG. 1 is a diagram showing left atrium 10, right pulmonary vein 20, left pulmonary vein 30, pericardium 40, and esophagus 50. As shown, esophagus 50 is in close proximity to left atrium 10, and thus is at risk of damage as a result of cardiac procedures such as atrial ablation. The devices and methods provided herein can reduce the likelihood (e.g., prevent) of esophageal damage and development of left atria-esophageal fistula, an often fatal complication that can occur as a consequence of atrial ablation.

The devices provided herein can include an elongate tubular member (e.g., a catheter) having a first end and a second end. The elongate tubular member can have any suitable length. For example, the elongate tubular member can have a length from about 20 cm to about 75 cm (e.g., 25 to 65 cm, 30 to 60 cm, 30 to 50 cm, 35 to 55 cm, or 40 to 50 cm) and a diameter from about 0.1 cm to about 0.7 cm (e.g., 0.2 to 0.6 cm, or 0.3 to 0.5 cm).

The elongate tubular member can define one or more lumens extending between the first and second ends. For example, a device can define a first lumen that extends from a point at or near the first end of the elongate tubular member to a point at or near the second end of the elongate tubular member. The first lumen can be configured such that a wire (e.g., a guidewire) can extend through the elongate tubular member via the lumen.

The devices can have an inflatable sleeve attached to the outer surface of the elongate tubular member. The inflatable sleeve can partially or completely surround the outer surface of a segment of the elongate tubular member. The inflatable sleeve typically is located at or near an end of the elongate tubular member.

The devices can define a second lumen between a point at the end opposite the inflatable sleeve and a point near the end at which the inflatable sleeve is located. The second lumen can open through the wall of the elongate tubular member such that it is in fluid communication with the interior of the inflatable sleeve. A fluid can be injected into the inflatable sleeve via the second lumen. In some embodiments, the end of the second lumen opposite the inflatable sleeve can be connected to an injection port and/or a valve, which can faciliate transfer of a fluid into and out of the interior of the inflatable sleeve.

The inflatable sleeve can be made of any suitable material. For example, when used in conjunction with an ablation device that can generate tissue heat of 50-60° C., the inflatable sleeve can be made of a material capable of withstanding heat of at least 50° C. (e.g., at least 50° C., at least 55° C., at least 60° C., at least 65° C., at least 70° C., or at least 100° C.). Such materials include, without limitation, polyethylene, polyurethane, and silicone thin film.

The inflatable sleeve can have any size. For example, the sleeve can have a length from 1.5 to 6.5 cm (e.g., 2 to 6 cm, 2.5 to 5.5 cm, 3 to 5 cm, or 3.5 to 4.5 cm), and a thickness from 0.3 to 1.2 cm (e.g., 0.4 to 1.1 cm, 0.5 to 1 cm, 0.6 to 0.9 cm, or 0.7 to 0.8 cm). When uninflated and flattened, the sleeve can have a width from 1 to 5 cm wide (e.g., 1.5 to 4.5 cm, 2 to 4 cm, 2.5 to 3.5 cm). In some embodiments, the sleeve can have a width less than 1 cm (e.g., less than 0.9 cm, 0.75 cm, 0.5 cm, or 0.25 cm). When inflated, the sleeve can have a diameter from about 0.3 cm to about 1.2 cm (e.g., 0.4 cm to 1.1 cm, 0.5 cm to 1 cm, 0.6 cm to 0.9 cm, or 0.7 cm to 0.8 cm). In addition, the inflatable sleeve can be adapted to take any suitable form. For example, when inflated, the inflatable sleeve can have a cross-section that is circular, oval, or in the shape of a crest.

In some embodiments, the inflatable sleeve can be made from or coated with (e.g., partially or entirely coated with) an echogenic material or an X-ray transparent material (i.e., a material that cannot pass X-rays and therefore is visible under fluoroscopy). Such a material (e.g., a porous material, a metallic material, or a metal oxide) can permit ultrasound and/or X-ray visualization during placement and/or inflation of the inflatable sleeve.

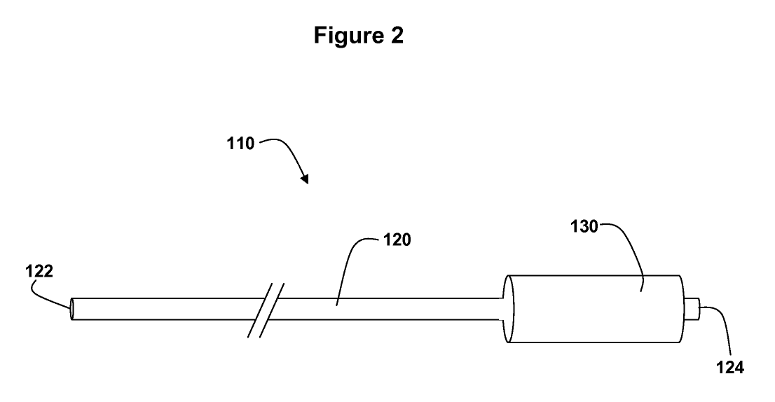

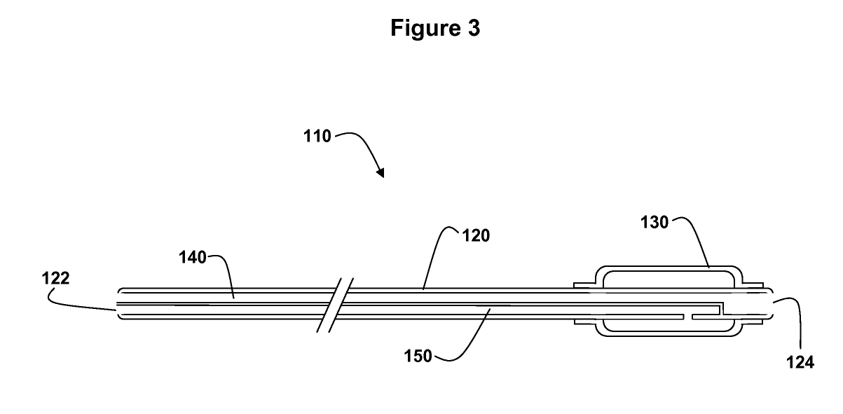

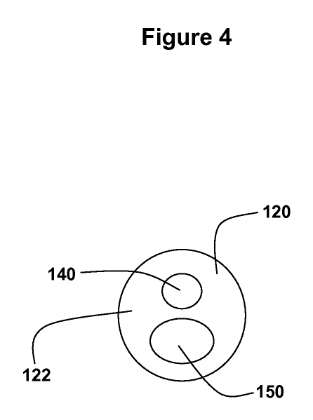

An example of a device as provided herein is shown in FIGS. 2, 3, and 4. With reference to these figures, device 110 can include catheter 120 having first end 122 and second end 124. Catheter 120 can be surrounded by balloon 130 at or near second end 124. Catheter 120 can have a plurality of lumens extending from about first end 122 to about second end 124. For example, catheter 120 can have first lumen 140 extending from first end 122 to second end 124. Lumen 140 can be configured such that a guidewire can be positioned within lumen 140 to extend from first end 122 to second end 124 of catheter 120. Catheter 120 can have second lumen 150 extending from first end 122 to the interior of balloon 130 at second end 124. Lumen 150 can be configured such that a fluid (e.g., a gas, a liquid, or a gel) can be passed via lumen 150 from first end 122 of catheter 120 into balloon 130, resulting in inflation of balloon 130.

This document also provides methods for using the devices provided herein. In general, a device provided herein can be positioned such that the inflatable sleeve is located within the pericardial space, between the esophagus and an atrium (e.g., the left atrium) of the heart. The sleeve can be inflated, and can protect the esophagus from thermotic or mechanical damage during a subsequent atrial ablation procedure.

The methods provided herein can include the insertion of a portion of a guidewire (e.g., an end of a guidewire) in the pericardial space. Any suitable method can be used to insert the guidewire into the pericardial space. In some embodiments, for example, the pericardial space can be accessed through a transthoracic pericardiocentesis method, such that the pericardium is punctured. In such procedures, an incision can be made into the chest cavity, and an endoscope can be inserted for visualization of the pericardium. The endoscope, or a puncturing instrument passed through the endoscope, can be positioned at a point proximate the surface of the pericardium (e.g., over the anterior surface of the pericardium). The pericardium then can be punctured using, for example, a needle passed through a lumen of the endoscope. A guidewire then can be inserted through the endoscope and into the pericardial space.

In some embodiments, the pericardial space can be accessed using a transvenous method. For example, a catheter can be inserted into a peripheral vein (e.g., a femoral vein or a jugular vein) and advanced through the vein to one of the vena cavae, through the vena cava to the right atrium, and subsequently into the right auricle. The catheter can be advanced into the apex of the right auricle so that a distal end of the catheter is placed against the wall of the right auricle. A guidewire can be passed through a lumen of the catheter and into the right auricle, and the wall of the auricle then can be pierced by the distal end of the guidewire, such that the end of the guidewire extends into the pericardial space. In some embodiments, transseptal catheterization can be used to access the left atrium.

Once the guidewire is inserted such that its distal end is positioned in the pericardial space, a device provided herein can be used. The elongate tubular member can be passed over the guidewire via the first lumen, such that the inflatable sleeve is advanced into the pericardial space. Ultrasound, fluorescence, or any other suitable technique can be used to monitor the position of the inflatable sleeve. Once the inflatable sleeve is positioned between the esophagus and the heart (e.g., the left atrium), a fluid can be passed through the second lumen and into the sleeve. Any suitable fluid can be used, including liquids (e.g., water or saline), gasses (e.g., air or oxygen), or gels. Inflation of the sleeve can effectively separate the esophagus from the heart, preventing or reducing the risk of esophageal damage as a result of a subsequent atrial ablation procedure. After the ablation procedure, the fluid can be removed from the sleeve via the second lumen such that the sleeve deflates, and the device can be removed from the body.

Other uses for the devices disclosed herein include, without limitation, protection of blood vessels and phrenic nerves from damage during ventricular arrhythmia ablation procedures. In addition, the devices can be used to protect organs (e.g., liver, spleen, uterus, ovaries, or lungs) or tissue during surgery or other procedures.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for protecting the esophagus during an atrial ablation procedure, comprising inserting a balloon into the pericardial space between the esophagus and the heart of a patient in need of an atrial ablation procedure, and inflating said balloon, wherein said balloon contains an echogenic or X-ray transparent material.

2. The method of claim 1, wherein said balloon is attached to a catheter.

3. The method of claim 2, wherein said balloon is in fluid communication with a lumen of said catheter.

4. The method of claim 1, wherein said balloon is inflated with air, water, saline, or gel.

5. The method of claim 1, wherein said balloon has a length between about 3 cm and about 5 cm.

6. The method of claim 1, wherein said balloon, when not inflated, has a width between about 2 cm and about 4 cm.

7. The method of claim 1, wherein said balloon, when inflated, has a diameter from about 0.5 cm to about 1 cm.

8. The method of claim 1, wherein said balloon has a thickness between about 0.3 cm and about 1.2 cm.

9. The method of claim 1, wherein said balloon is made of a material capable of withstanding heat of at least 50° C.
